(19) United States
(12) Patent Application Publication
(10) Pub. No.: US 2024/0057633 A1
(43) Pub. Date: Feb. 22, 2024

(54) Hollow candy for delivering liquid medicine for human consumption

(71) Applicant: Jessica Agostinis, Tampa, FL (US)

(72) Inventor: Jessica Agostinis, Tampa, FL (US)

(21) Appl. No.: 17/888,484

(22) Filed: Aug. 16, 2022

Related U.S. Application Data
(60) Provisional application No. 63/237,985, filed on Aug. 27, 2021.

(51) Int. Cl. A23G 3/34; A23L 29/231; A23L 29/256; A23L 29/281; A61K 47/69; A61K 9/00

(52) U.S. Cl. CPC A23G 3/0053; A23L 29/231; A23L 29/256; A23L 29/281; A61K 47/6939; A61K 9/0056

(57) ABSTRACT

Confectionery products such as candies where a homogenous mixture of hard sugar-based coating or chewable soft coating have an inner hollow pouch to infuse medicines and a central vein to lead from a top to a central cavity or pouch. A preferred combination of sweet and fruit flavor profiles with the prescribed medicine hidden within it, provides a confection which is attractive for children, and is crispier, fresh and crunchier once consumed by the user.

A portion of the disclosure of this patent document contains material which is subject to copyright protection. The copyright owner has no objection to the facsimile reproduction by anyone of the patent disclosure, as it appears in the Patent and Trademark Office patent files or records, hut otherwise reserves all copyright rights whatsoever.

### BACKGROUND

### Field of the Invention

This invention relates to a confectionery incorporated with central cavity to induce and conceal medicine. Confectionery herein after described includes primarily candies but can include chocolates and biscuits and the like products. The central candy part is hollow for delivering and disguising the child's liquid medicine or tablets and deliver it in a fun and tasty manner.

### Description of the Related Art

The present invention is generally directed to medications and more specifically to a candy with central pouch for storing medication and a method for administering the same. The central hollow pouch could also be used for more recreational uses like to be filled with chocolate/candy syrups, liqueurs, etc.

Administering medications to a sick child has been and continues to be one of the most unpleasant tasks a parent must undertake. There are many factors that contribute to the difficulty in making a child take medication. For example, most children have a fear of doctors, drugs and medicine in general. In addition, many children have difficulty swallowing many medications in either liquid or tablet form because of a gag reflex caused by a mental phobia and taste aversion to many medications.

However, as proclaimed in the motion picture Mary Poppins, “Just a spoon full of sugar makes the medicine go down in the most delightful way.” in an attempt to exploit this theory, several attempts have been made to alleviate the fears and anxiety for children taking medications by somehow sweetening the medication. A common example is to provide a type of fruit or candy flavoring to a liquid medication, such as flavored cough syrups.

Also, efforts to make medications for children fun to ingest have been attempted. For example, chewable vitamins having fruit flavors play to a child's love of candy. Also, many such medications are shaped in the form of a cartoon character, an animal, etc.

However, the known attempts to cater to children's likes do not provide a more effective way of actually administering the medication itself. For example, the fact that the medication tastes like candy does not aid in the physical ingestion of the medication.

Accordingly, a need has arisen for a highly-dissolvable candy medication that facilitates physical ingestion of the medication by the patient. In addition, the related need for a method of making and administering such a medication also exists.

This invention also relates to a process of producing confectionery comprising the steps of preparing confectionery mixture incorporating the central hollow area for adding liquid or tablet medicine and sugar-based hard outer coating. This invention will herein after be described with reference to the production of candy having a hollow center to be infused with medicine by caregivers at later stage or and a sugar-based hard or chewable soft coating. It is to be understood that the following examples do not exclude other confectionery and their processes of production. The example does not restrict the appended claims in any manner and are only illustrative.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

in light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved form of a technique for making a candy consisting of a hollow center for adding medicine and a sugar-based hard thin coating or soft chewable coating which has a stable multi-layer structure. The vein or canal will lead to the cavity from a hole in the top of the gummy, so that parents can pinch the opening closed once medicine is inserted to keep from leaking. It can also include no opening in the top but a slit in the cavity wall for inserting tablets.

The main objective of the invention is to provide stress-free delivery to the child with a fun character, in a gummy form that kids will love and will consume effectively.

It is further the objective of the invention to provide Gummy with ingredients including but not limited to gelatin/agar agar/pectin fruit puree-based fruit candy cavity/pouch with hollow centers with a hole in the top for inserting liquid filled syringe.

It is also the primary objective of the invention to provide a vein connecting the cavity to the hole for inserting liquid filled syringe and parents can pinch the opening closed once medicine is inserted to keep from leaking or slit in center for inserting and disguising tablets.

It is also the objective of invention to provide Gummy texture malleable enough to pinch injection hole back shut with fingers after to seal in the liquid.

Thus, it is the objective to provide a new and improved candy arrangement with hollow center to infuse medicine. Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention and explain various principles and advantages of those embodiments.

FIG. 1 is a front view of option 1 as per preferred embodiments of the invention.

FIG. 2 is a side view of option 1 as per preferred embodiments of the invention.

FIG. 3 is a front view of option 2 as per preferred embodiments of the invention.

FIG. 4 is a side view of option 2 as per preferred embodiments of the invention.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present invention is directed to a new concept combining a centered central hollow pouch for adding medicine with a soft malleable gummy with the cavity and canal, so that parents can pinch the opening closed once medicine is inserted to keep from leaking. Thus, it will be a sturdy/structural Gummy/fruit snack/jelly wall with a cavity in the middle in vein leading in/out or a slit for insertion and removal of tablets.

The invention as per its preferred embodiments includes (1) gummy candy cavity with small hole in top is held by adult in one hand and with the other hand inserts liquid from medicine filled syringe (2), releasing liquid into the cavity where adult can then remove the syringe and (3) pinch the hole at the top of the malleable gummy shut to seal in liquid and deliver to child. Another variation of this could be a hollow gummy candy cavity with no hole exposed for syringe but rather the syringe is poked through a portion of the gummy exterior wall into the cavity where liquid is then released. The gummy candy could be rolled in citric acid and a sweetener to make a sour candy option. Different healthy benefits could be added to the gummy candy mixture like probiotics, vitamin C, elderberry syrup for immunity boosting, electrolytes, or using juice instead of fruit puree. Another variation of this product could be more of a recreational gummy candy treat, not necessarily marketed for sick children, if injected with chocolate for chocolate filled gummies, or an adult treat of alcohol/liqueur filled gummies. These could be with the (2) design of a hole left in the top, or completely closed off for injecting through the barrier wall or prefilled with recreational chocolate/liquid in production. The candy can also have a splice in the center of the gummy where the cavity is for parents to insert medicine in tablet form.

The typical formula for discussed candy includes:

| | |
|---|---|
| Sugar | 25-40% |
| Corn Syrup | 50-65% |
| Cooking Starch | 10% |
| Gelatin | <2% |
| Flavorings | <1% |
| Coloring | <1% |

Use a gelatin, pectin, or agar agar and a fruit puree-based recipe to cook the gummy candy mixture. This step along with the ones to follow can be done at home on a stovetop, in a commercial kitchen, or outsourced to a candy or food/product manufacturer. Pour completed mixture into 3D silicon mold and using slush casting, rotational casting, or blow molding rotate until outer walls of the gummy candy have set, remove from mold, roll in nonstick agent such as monk fruit powder or corn starch, and package individually.

All elements are necessary for the production of the hollow candy gummy. Rolling completed gummy in citric acid and monk fruit sweetener could work better for an extra nonstick coating and sour candy flavor, which may mask a medicine taste even more. Using 100% fruit juice instead of 100% fruit puree could result in a gummy candy with a more ideal appearance and consistency.

If no hole were left in the top of this gummy candy someone could potentially use a syringe to simply puncture a hole through a gummy wall into the cavity. Someone could make a gummy candy broken into two halves and when an adult pours the liquid into one half of the exposed cavity, they could lay the remaining half on top and pinch around the perimeter of the gummy sealing it shut, similar to crimping an empanada or pie crust. Gummy candy could be made into an open cylinder shape, liquid could be poured in and then the top could still be pinched shut to seal. Furthermore, the splice in the center wall of the cavity can also be provided for parents to insert tablet/s.

The process of use this invention to insert tablet via slice into cavity or to inject liquid medicine using a medicine syringe into the hollow gummy candy and seal off the top by pinching shut with one's fingers and deliver to sick child for a fun medicine taking experience with the added benefits of additional flavor kids love. This invention as per its further embodiments could also be used to inject non-pharmaceutical liquids, such as inched chocolate for a tasty candy treat, or a liqueur for adult consumption.

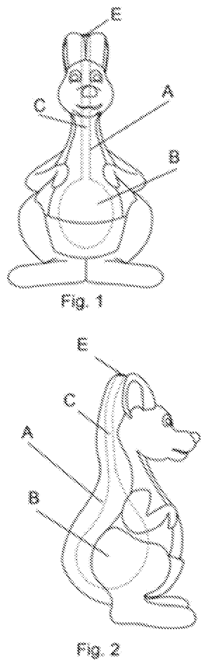

By looking at provided drawings, the structure of candy can be seen. The FIG. 1 is a front view of option 1 where Gummy/fruit snack (A), Liquid medicine compartment (B), Vein to insert medicine (C) and opening to insert syringe (F) can be seen. FIG. 2 is a side view of option 1 as per preferred embodiments of the invention and discloses Gummy/fruit snack (A), Liquid medicine compartment (B), Vein to insert medicine (C) and opening to insert syringe (F) can be seen.

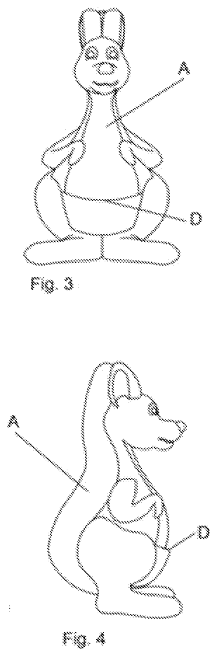

FIG. 3 is a front view of option 2 as per preferred embodiments of the invention and FIG. 4 is a side view of option 2 as per preferred embodiments of the invention. In these figures Gummy/fruit snack (A) and Slit to insert chewable tablet in pouch (D) can be seen.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1-2. (canceled)

3. A hollow confection for administration of a medication comprising:
a gummy candy composition having a wall,
a central cavity or hollow pouch within the wall for receiving and holding a medication, and
a vein or canal connecting an injection hole in the wall to the central cavity or hollow pouch, for inserting a liquid medication filled syringe through the injection hole into the vein or canal in the wall for injection of the liquid medication through the wall into the central cavity or hollow pouch, or
a slit or splice in the wall for insertion of a medication in tablet form into the central cavity or hollow pouch,
wherein the gummy candy composition is malleable so that the injection hole, slit or splice in the wall can be pinched shut with fingers to seal the liquid medication or tablet within the central cavity or hollow pouch of the gummy candy composition, and wherein the gummy candy composition is highly dissolvable to facilitate the physical ingestion of the medication by a patient.

4. A confection for administration of a medication as claimed in claim 3 having a vein or canal connecting an injection hole in the wall to the central cavity or hollow pouch, for inserting a liquid medication filled syringe through the injection hole into the vein or canal in the wall for injection of the liquid medication through the wall into the central cavity or hollow pouch, and the gummy candy composition is malleable so that the injection hole in the wall can be pinched shut with fingers to seal the liquid medication within the central cavity or hollow pouch of the gummy candy composition to prevent liquid from pouring back out.

5. A confection for administration of a medication as claimed in claim 3 having a slit or splice in the wall for insertion of a medication in tablet form into the central cavity or hollow pouch.

6. A confection for administration of a medication as claimed in claim 3 having a sugar based hard or soft chewable coating.

7. A confection for administration of a medication as claimed in claim 3 in the form of a character.

8. A confection for administration of a medication as claimed in claim 4 in the form of a kangaroo character, wherein the injection hole and vein are in a tail of the kangaroo, and the cavity or hollow pouch are in the belly of the kangaroo.

9. A confection for administration of a medication as claimed in claim 5 in the form of a kangaroo character, wherein the central cavity or hollow pouch is in the belly of the kangaroo, and the slit or splice in the wall for insertion of a medication in tablet form is a slice in the belly of the kangaroo which allows placement of the tablet by tucking into the central cavity or hollow pouch.

10. A confection for administration of a medication as claimed in claim 3 wherein the gummy candy composition comprises gelatin, agar agar, pectin, fruit puree, or fruit juice.

11. A confection for administration of a medication as claimed in claim 3 wherein the gummy candy composition comprises 25-40% sugar, 50-65% corn syrup, 10% cooking starch, less than 2% gelatin, less than 1% flavoring, and less than 1% coloring.

12. A confection for administration of a medication as claimed in claim 3 which is individually packaged for subsequent insertion of the medication into the central cavity or hollow pouch.

13. A method for preparing the hollow confection for administration of a medication claimed in claim 3 comprising:
cooking a mixture of gelatin, pectin, or agar agar with a fruit puree or fruit juice to obtain a gummy candy composition,
pouring the gummy candy composition into a 3D silicon mold,
subjecting the mixture to slush casting, rotational casting, or blow molding rotation until outer walls of the gummy candy composition have set,
removing the gummy candy composition from the mold,
rolling the molded gummy candy composition in a nonstick agent, and
individually packaging the hollow confection.

14. A method for preparing the hollow confection for administration of a medication as claimed in claim 13 wherein the confection has a vein or canal connecting an injection hole in the wall to the central cavity or hollow pouch, for inserting a liquid medication filled syringe through the injection hole into the vein or canal in the wall for injection of the liquid medication through the wall into the central cavity or hollow pouch, and the gummy candy composition is malleable so that the injection hole in the wall can be pinched shut with fingers to seal the liquid medication within the central cavity or hollow pouch of the gummy candy composition to prevent liquid from pouring back out.

15. A method for preparing the hollow confection for administration of a medication as claimed in claim 13 wherein the confection has a slit or splice in the wall for insertion of a medication in tablet form into the central cavity or hollow pouch.

16. A method for preparing the hollow confection for administration of a medication as claimed in claim 13 wherein the confection has a sugar based hard or soft chewable coating.

17. A method for preparing the hollow confection for administration of a medication as claimed in claim 13 wherein the confection is in the form of a character.

18. A method for preparing the hollow confection for administration of a medication as claimed in claim 14 wherein the confection is in the form of a kangaroo character, wherein the injection hole and vein are in a tail of the kangaroo, and the cavity or hollow pouch are in the belly of the kangaroo.

19. A method for preparing the hollow confection for administration of a medication as claimed in claim 15 wherein the confection is in the form of a kangaroo character, wherein the central cavity or hollow pouch is in the belly of the kangaroo, and the slit or splice in the wall for insertion of a medication in tablet form is a slice in the belly of the kangaroo which allows placement of the tablet by tucking into the central cavity or hollow pouch.

20. A method for preparing the hollow confection for administration of a medication as claimed in claim 13 wherein the gummy candy composition comprises 25-40% sugar, 50-65% corn syrup, 10% cooking starch, less than 2% gelatin, less than 1% flavoring, and less than 1% coloring.
